(19) United States
(12) Patent Application Publication
(10) Pub. No.: US 2007/0086990 A1
(43) Pub. Date: Apr. 19, 2007

(54) Table olives containing probiotic microorganisms

(75) Inventors: Paola Lavermicocca, Bari (IT); Stella Lisa Lonigro, Adelfia-Bari (IT); Angelo Visconti, Bari (IT); Maria De Angelis, Teramo (IT); Francesca Valerio, Bari (IT)

Correspondence Address: KENYON & KENYON LLP, ONE BROADWAY, NEW YORK, NY, 10004, US

(73) Assignee: Consiglio Nazionale Delle Ricerche, Roma (IT)

(21) Appl. No.: 10/581,516

(22) PCT Filed: Nov. 30, 2004
(86) PCT No.: PCT/EP04/13582

§ 371 Date: Dec. 13, 2006

(30) Foreign Application Priority Data
Dec. 5, 2003 (IT) MI2003A002391

(51) Int. Cl. A61K 35/74; A61K 36/63; A23B 7/10

(52) U.S. Cl. 424/93.45; 426/49; 424/769

(57) ABSTRACT

The invention relates to table olives enriched with probiotic microorganisms, in particular lactobacilli and bifidobacteria, food products containing them and a method for their preparation. The olives and food products of the invention provide an amount of microorganisms sufficient to exert a beneficial action on the gastro-intestinal tract and are particularly advantageous whenever administration of probotic food products of animal origin, in particular milk derivatives, is not possible.

### FIELD OF THE INVENTION

The present invention relates to probiotic food products, i.e. food products containing microorganisms having a beneficial effect on health, in particular on the gastrointestinal tract.

### BACKGROUND OF THE INVENTION

Probiotic food products are in general fermented foods containing an amount of viable and active microorganisms large enough to reach the intestine and exert an equilibrating action on the intestinal microflora.

Intake of probiotics stimulates the growth of beneficial microorganisms, reduces the amount of pathogens and strengthens the body's natural defences. It is acknowledged that probiotic bacteria, in particular lactobacilli and bifidobacteria, help to maintain the equilibrium of the intestinal flora (Salminen S., et al. Int. Dairy J. 8:563-572, 1998; Saarela M., L. et al., Int. J. Food Microbiol. 2002, 78:99-117) and inhibit pathogens (Drago L., M. R. et al., FEMS Microbiol. Letters, 1997, 153:455-463 and Cross M. L. FEMS Immunol. Med. Microbiol. 2002, 34:245-253), thus lowering the risk of gastro-intestinal diseases. In fact, when the intestinal microflora is altered, administration of probiotic bacteria not only re-establishes its normal equilibrium, but also improves the microbial balance and properties of the endogenous flora. The role of probiotics in the prevention of food allergies and intolerances is also under study (Isolauri E., et al., Am. J. Clin. Nutr. 2001, 73 (suppl.): 444s-450s; Jahreis G., et al. Food Res. Int. 2002, 35:133-138).

Probiotic bacteria are introduced in food products for human nutrition, especially in fermented milk, for example in yogurt. One of the problems related to the production of probiotic foods is the influence of production technologies on strains properties, in particular cell viability, integrity, and population stability (Mattila-Sandholm T., et al. Int. Dairy, 2002 J. 12:173-182). Liquid and frozen cultures were largely used in the past, but their production, transport and storage costs are high. Lyophilized cultures are presently widespread, but cells are often damaged and cannot be stored for a long time. In fact lyophilised cells survive in anaerobiosis and viability is restored by rehydration. This treatment not only does not ensure survival of all the cells, but the survived ones may also be metabolically altered and not withstand gastric acidity. Concentrated monodose cell cultures are also widespread. In this case the greatest difficulty is to reach high cell concentrations, i.e. to about 10¹⁰ (UFC)/g. Therefore, most of the presently available probiotics are of animal origin, in particular dairy products, such as yogurt, cheese, desserts, ice-creams. However, dairy product consumption may be limited due to allergies or intolerances to milk and derivatives thereof. Also known is the difficulty of introducing bifidobacteria—largely used in probiotics—in fermented milk products, due to their strain-related sensibility to milk-fermenting bacteria, pH, temperature and oxygen concentration (Gobbetti M. et al. J. Dairy Sci. 1998, 81:37-47).

Probiotic dehydrated fruits have been obtained on an experimental scale by vacuum drying fruits soaked in probiotic microorganisms (Betoret N., et al. J. Food Engin. 2003, 56:273-277), while some oat-based products and fruit juices containing probiotic bacteria are already available on the market (Johansson et al. Int. J. Food Microbiol., 1998, 42:29-38).

It should also be pointed out that all the above-mentioned products must be consumed rapidly after opening.

It should therefore be advantageous to provide food products that allow to administer probiotic bacteria without causing allergies or intolerances and that can be stored for a long time after opening.

### DETAILED DESCRIPTION OF THE INVENTION

The present invention relates to probiotic food products based on table olives containing probiotic bacteria.

In a first embodiment, the food product consists of table olives whose pericarp is coated with microorganisms of the Lactobacillus and Bifidobacterium genus, in particular probiotic lactobacilli and bifidobacteria. Preferably, the lactobacilli are selected from Lactobacillus rhamnosus and Lactobacillus paracasei, while the bifidobacteria are selected from Bifidobacterium bifidum and Bifidobacterium longum. Even more preferably, the microorganisms are selected from: Lactobacillus rhamnosus GG ATCC53103; L. rhamnosus IMPC 11; L. rhamnosus IMPC 19; Lactobacillus paracasei IMPC 2.1 (deposited with the Belgian Coordinated Collections of Microorganisms, BCCM/LMG-Collection, Gent, Belgium, under accession number LMG P-22043); Lactobacillus paracasei IMPC 4.1; Bifidobacterium bifidum ATCC15696 and Bifidobacterium longum ATCC15708.

The olives of the invention can be prepared by keeping table olives in a suspension of the desired microorganism, at room temperature (about 25° C.), thus obtaining olives on whose pericarp microorganisms adhere in amounts ranging from 5×10⁵ to 5×10⁸ UFC/gram (evaluation after 3 month storage, see tables 1 and 2).

The table olives of the invention can be either consumed as such, or used for the preparation of probiotic food products, which are a further embodiment of the invention.

The olives and probiotic foods of the invention are an effective means to treat or prevent intestinal disorders or restore the intestinal flora after antibiotic therapy.

Particularly beneficial are the olives enriched with L. paracasei IMPC 2.1, not only due to the marked probiotic characteristics of this microorganism, its ability to grow both under aerobic and anaerobic conditions and adhere to the pericarp, but also due to its resistance to gastric juices and bile salts. L. paracasei IMPC 2.1 is a new microorganism and is a further embodiment of the invention.

Particularly important is also the possibility of incorporating bifidobacteria, since it is known that these microorganisms hardly grow and survive in fermented milk products.

The olives of the present invention and the food products containing them are particularly useful for the prevention and treatment of diseases caused by food contaminants, in gastro-intestinal diseases affecting travellers, as coadjuvants in antibiotic therapy and, more generally, in situations in which it is necessary to increase the body immune defences.

Thanks to convenient administration, storage in non-refrigerated conditions (after 90 days at room temperature the bacterial count ranges from 1×10⁵ to 7.6×10⁷ UFC per gram), as well as organoleptic properties, the olives can be consumed whenever prompt administration of probiotic bacteria is required, even by lactose-intolerant people. A further advantage is that consumption of only part of the package content (i.e. olives, not brine), provides a dose of probiotic bacteria that corresponds to that provided by yogurt or concentrated cultures.

Finally, it must also be pointed out that with respect of probiotic foods of animal or vegetal origin, wherein microorganisms are re-suspended in a liquid medium, in the case of olives the bacterial cells are immobilized, which ensures an effective, safe transport in the gastro-intestinal tract. Moreover, the binding to a product containing a large amount of fats, allows the microorganisms to resist to gastric juices.

### EXPERIMENTAL SECTION

### Example 1

Viability of Probiotic Bacteria on the Olive Pericarp

Colonization of the pericarp of table olives and the survival of the following strains have been evaluated: Lactobacillus rhamnosus GG ATCC53103, L. rhamnosus IMPC 11 and IMPC 19, Lactobacillus paracasei IMPC 2.1 and IMPC 4.1, Bifidobacterium bifidum ATCC15696 and Bifidobacterium longum ATCC15708.

Lactobacillus paracasei IMPC 2.1 was deposited with the Belgian Coordinated Collections of Microorganisms, BCCM/LMG-Collection, Gent, Belgio under accession number LMG P-22043.

Tests were carried out on stoned and whole black olives, previously de-bittered and processed so as to make them edible. The same tests were also carried out on fresh or semifinished green and black olives and on de-bittered and processed green olives (finished product). Strains viability was evaluated using jars containing 80 olives immersed in 280 ml of their own brine or in NaCl 4%±fructose 0.2÷1%, pH 6.5.

Procedure. Black olives immersed in their own brine (finished product) were added with a bacterial suspension containing from 4×10⁹ to 9×10¹⁰ (UFC) of each strain. After the inoculum the olives are placed in sterile jars closed with screw-caps. Non-inoculated olives, also in jars, were used as the control. The samples were stored for 3 months at room temperature (about 25° C.), thereafter 4 olives were taken from each sample at t=1, 15, 30 and 90 and submitted to bacterial count. Brine was thoroughly removed and the olives were added with 20 ml 0.85% NaCl and 0.025% Tween 80 and vigorously shaken for two hours to detach the bacteria from the pericarp. The resulting suspension was seeded on an agar substrate for the count of lactic bacteria. The results are reported in the following table.

TABLE 1
UFC per gram of stoned olives

| strain | 1 day | 15 days | 30 days | 90 days |
|---|---|---|---|---|
| L. rhamnosus GG | 5.0 × 10⁸ | 3.5 × 10⁷ | 3.4 × 10⁷ | 3.2 × 10⁷ |
| ATCC53103 | | | | |
| L. rhamnosus IMPC 11 | 7.0 × 10⁷ | 8.0 × 10⁸ | 7.5 × 10⁷ | 7.6 × 10⁷ |
| L. rhamnosus IMPC 19 | 7.2 × 10⁷ | 8.5 × 10⁸ | 6.8 × 10⁶ | 6.9 × 10⁶ |
| L. paracasei IMPC 2.1 | 7.6 × 10⁷ | 2.0 × 10⁹ | 7.9 × 10⁷ | 6.0 × 10⁶ |
| L. paracasei IMPC 4.1 | 4.8 × 10⁷ | 5.8 × 10⁷ | 6.5 × 10⁷ | 6.8 × 10⁷ |
| B. bifidum ATCC 15696 | 2.5 × 10⁷ | 8.0 × 10⁷ | 5.2 × 10⁷ | 4.3 × 10⁶ |
| B. longum ATCC 15708 | 4.5 × 10⁶ | 2.7 × 10⁷ | 8.7 × 10⁶ | 5.2 × 10⁵ |

TABLE 2
UFC per gram of whole olives

| strain | 1 day | 15 days | 30 days | 90 days |
|---|---|---|---|---|
| L. rhamnosus GG | 1.8 × 10⁷ | 2.3 × 10⁶ | 7.6 × 10⁵ | 3.9 × 10⁶ |
| ATCC53103 | | | | |
| L. rhamnosus IMPC 11 | 7.0 × 10⁶ | 1.0 × 10⁷ | 2.4 × 10⁶ | 1.5 × 10⁶ |
| L. rhamnosus IMPC 19 | 3.5 × 10⁶ | 1.0 × 10⁷ | 2.5 × 10⁵ | 2.7 × 10⁵ |
| L. paracasei IMPC 2.1 | 7.4 × 10⁶ | 3.0 × 10⁷ | 4.4 × 10⁷ | 9.0 × 10⁶ |
| L. paracasei IMPC 4.1 | 7.1 × 10⁶ | 1.1 × 10⁷ | 5.4 × 10⁷ | 8.0 × 10⁶ |
| B. bifidum ATCC 15696 | 1.3 × 10⁶ | 7 × 10⁶ | 3.6 × 10⁶ | 1.2 × 10⁶ |
| B. longum ATCC 15708 | 5.0 × 10⁵ | 3.6 × 10⁶ | 3.6 × 10⁶ | 1.0 × 10⁵ |

All the experiments were repeated twice and relevant variations were not observed.

The pericarp allows tight anchorage of the bacteria and ensures their slow release after intake, as demonstrated by the drastic re-suspension procedure. In particular, about 10⁶ UFC/g were recovered from samples analysed 30 days after addition of the bacteria by vigorous stirring for 2 hours in physiological solution added with Tween; after 3 subsequent washings (1 h each in the same conditions) about 10⁵, 10⁴ and 10³ UFC/9 g still adhered to the pericarp.

Similar tenacity was observed in samples taken after 7 or 90 days from the addition of the bacteria.

### Example 2

Selection of Lactobacillus paracasei IMPC 2.1 (Reference Strain)

Lactobacillus paracasei IMPC 2.1 was isolated from a healthy adult human subject with a bacterial population of 10⁷ UFC/ g in faeces.

Strain Genetic Identification

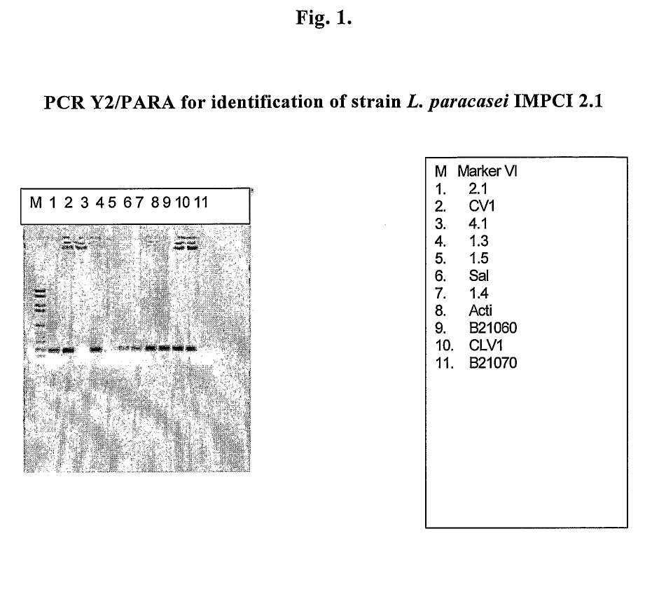

Species-specific PCR with Y2/PARA primers (FIG. 1) was carried out as the first identification step. Y2 is the universal primer for eubacteria, while PARA is the specific primer for L. paracasei. IMPC 2.1 showed an amplification band of 290 bp, typical of L. paracasei species.

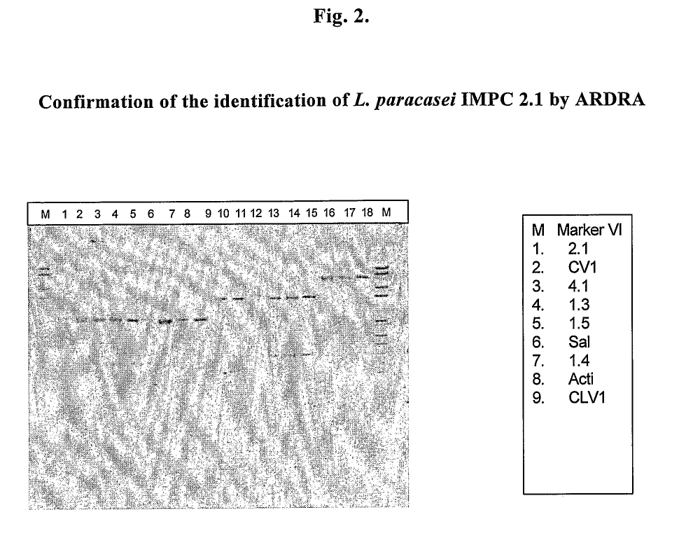

ARDRA using Sau 3AI as the restriction enzyme was carried out as a confirmation analysis; also in this case the expected restriction profiles of L. paracasei were obtained (FIG. 2).

L. paracasei IMPC 2.1 is able to tightly adhere to pig intestinal mucus, abiotic surfaces and pericarp and is highly resistant to bile acids, as demonstrated by the following experiments.

Adhesion to Pig Intestinal Mucus

An in vitro test for adhesion to pig intestinal mucus was carried out to evaluate in vivo adhesion, according to the method of Schou, et al. (APMIS 1999, 107: 493-504), partially modified as follows.

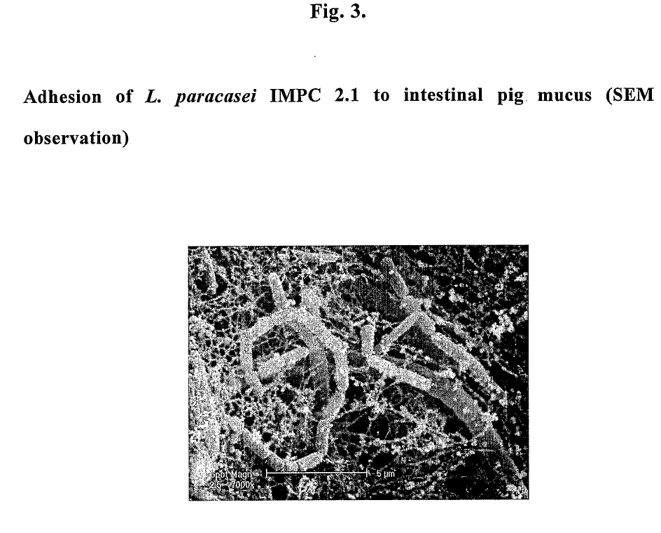

96-Well plates, coated with pig mucus (Tipe II, Sigma), were seeded with a titred bacterial suspension (100 μ1, PBS buffer). After incubation for 2 hrs at 37° C. with rocking, the plates were washed three times with PBS and the mucus was mechanically removed from the wells, then the washings and mucus were seeded in plates. FIG. 3 reports a SEM image of L. paracasei IMPC 2.1 adhering to the mucus after three washings.

The L. paracasei strains used in the test are listed hereinbelow, together with the results of the count (percentage ratio of UFC on the mucus in the final step to UFC in the titred bacterial suspension)

1) IMPC 2.1=40%

2) IMPC CV1=37%

3) IMPC 4.1=10%

4) IMPC 1.3=40%

5) IMPC 1.5=33%

6) IMPC 1.4=35%

7) Chr.Hansen Lc1=39%

8) IMPC CLV1=38%

9) ATCC 10863=18%

IMPC 2.1 is one of the strains which adhere better.

Resistance to Bile Salts

The resistance of L. paracasei strains was evaluated using MRS medium (De Man et al., J. Appl. Bacteriol., 1960, 23:130-135) containing Oxgall bovine bile salts at different concentrations. The first tests were carried out using 0.2, 0.3, 0.4% Oxgall: in these conditions the strain showed slightly reduced growth at increased concentrations. Growth was evaluated by measuring optical density (OD) at 600 nm.

| Strains | MRS | 0.2% Oxgall | 0.3% Oxgall | 0.4% Oxgall |
|---|---|---|---|---|
| 2.1 | 1.847 | 1.678 | 1.739 | 1.570 |
| Acti | 1.942 | 1.587 | 1.314 | 1.043 |
| Sal | 1.942 | 1.674 | 1.583 | 1.451 |
| CV1 | 1.853 | 1.640 | 1.518 | 1.312 |
| CLV1 | 1.813 | 1.688 | 1.634 | 1.344 |
| B21070 | 1.714 | 1.455 | 1.316 | 1.185 |
| B21060 | 1.954 | 1.789 | 1.657 | 1.453 |
| 1.3 | 1.829 | 1.818 | 1.697 | 1.583 |
| 1.4 | 1.843 | 1.840 | 1.679 | 1.581 |
| 1.5 | 1.875 | 1.760 | 1.818 | 1.674 |
| 4.1 | 1.978 | 1.694 | 1.441 | 1.559 |

In the subsequent step (the bile acid) concentration was increased up to 0.7%.

| Strain | MRS | 0.5% Oxgall | 0.6% Oxgall | 0.7% Oxgall |
|---|---|---|---|---|
| 2.1 | 1.458 | 0.792 | 0.178 | 0.095 |
| Acti | 1.548 | 0.139 | 0.061 | −0.132 |
| Sal | 1.354 | 0.758 | 0.562 | 0.353 |
| CV1 | 1.399 | 0.160 | −0.038 | −0.156 |
| CLV1 | 1.313 | 0.322 | 0.176 | 0.055 |
| B21070 | 1.435 | −0.142 | −0.200 | −0.193 |
| B21060 | 1.367 | 0.729 | 0.611 | 0.280 |
| 1.3 | 1.377 | 0.576 | 0.234 | 0.314 |
| 1.4 | 1.525 | 0.695 | 0.927 | 0.396 |
| 1.5 | 1.475 | 0.866 | 0.916 | 0.603 |
| 4.1 | 1.502 | 0.817 | 0.764 | 0.561 |

IMPC 2.1 proved one of the strains with good resistance to bile salts.

Salinity Resistance

MRS medium was used to evaluate strain resistance to different NaCl concentrations. Also in this case growth was evaluated by measuring optical density (OD) at 600 nm.

| Strain | MRS | 0.5% NaCl | 1% NaCl | 2% NaCl |
|---|---|---|---|---|
| 2.1 | 1.847 | 1.644 | 1.457 | 1.513 |
| Acti | 1.942 | 1.551 | 1.689 | 1.483 |
| Sal | 1.942 | 1.685 | 1.665 | 1.601 |
| CV1 | 1.853 | 1.555 | 1.541 | 1.781 |
| CLV1 | 1.813 | 1.512 | 1.689 | 1.648 |
| B21070 | 1.714 | 1.711 | 1.658 | 1.491 |
| B21060 | 1.954 | 1.560 | 1.717 | 1.510 |
| 1.3 | 1.829 | 1.656 | 1.631 | 1.697 |
| 1.4 | 1.843 | 1.658 | 1.795 | 1.737 |
| 1.5 | 1.875 | 1.534 | 1.554 | 1.697 |
| 4.1 | 1.978 | 1.811 | 1.762 | 1.596 |

Since high growth rate was observed also with 2% NaCl, tests with higher concentrations were carried out.

| Strain | MRS | 3% NaCl | 4% NaCl | 5% NaCl |
|---|---|---|---|---|
| 2.1 | 1.300 | 1.217 | 1.081 | 0.896 |
| Acti | 1.327 | 1.317 | 1.169 | 1.073 |
| Sal | 1.288 | 1.275 | 1.180 | 0.985 |
| CV1 | 1.283 | 1.185 | 1.031 | 0.829 |
| CLV1 | 1.217 | 1.158 | 1.036 | 0.799 |
| B21070 | 1.266 | 1.269 | 1.128 | 0.947 |
| B21060 | 1.321 | 1.177 | 1.090 | 0.962 |
| 1.3 | 1.239 | 1.207 | 1.050 | 0.922 |
| 1.4 | 1.306 | 1.183 | 1.026 | 0.823 |
| 1.5 | 1.289 | 1.266 | 1.061 | 0.899 |
| 4.1 | 1.291 | 1.245 | 1.075 | 0.897 |

IMPC 2.1 proved to be one of the strains with better salinity resistance.

Resistance to Simulated Gastric Juice (UFC/ml)

Strain resistance to simulated gastric juice was evaluated using different strains cultured in MRS medium. The cultures were washed with sterile saline and added to an equal volume of simulated gastric juice (NaCl, 125mM⁻¹; KCl 7 mM⁻¹; NaHCO₃, 45 mM⁻¹ and pepsin, 3 gr l⁻¹), adjusting the pH to 2 with HCl. The suspensions were then incubated at room temperature under stirring (200 rev min⁻¹) to simulate peristalsis. Aliquots were taken at time 0 and after 90 and 150 minutes and counted on MRS agar.

| Strain | T₀ | T₁(90 min) | T₂(150 min) |
|---|---|---|---|
| 2.1 | 44 · 10⁶ | 1.22 · 10⁸ | 2.15 · 10⁷ |
| 1.4 | 41 · 10⁶ | 4.5 · 10⁷ | 3 · 10⁷ |
| B21070 | 17.8 · 10⁶ | 2 · 10⁷ | 5.8 · 10⁷ |
| Sal | 197 · 10⁶ | 1.13 · 10⁸ | 5.2 · 10⁷ |
| 4.1 | 116 · 10⁶ | 1.09 · 10⁸ | 1.77 · 10⁷ |

IMPC 2.1 proved one of the strains with better resistance to simulated gastric juice.

Adhesion to Abiotic Surfaces

Adhesion ability, necessary for the strains to colonize the intestinal mucosa, was evaluated also with a test for adhesion to abiotic surfaces (Tuomola et al., Int. J. Food Microbiol., 2000, 41:45-51). The strains were cultured in MRS medium, at 37° C. for 48 hours under anaerobiosis. The cultures were then diluted 1:40 in MRS and 200 μl aliquots were seeded in 96-well polystyrene plates. After incubation for 24 hours at 37° C. the wells were gently rinsed with Dulbecco's phosphate buffer (DPBS, pH 7.3), allowed to dry and added with a crystal violet solution to stain the cells. Excess of dye was washed away with ethanol-acetone (80:20 v/v), then optical density (DO) was measured with an automatic reader. On the basis of DO values, cells were divided into 4 adhesion classes: no adhesion (AC1, OD≦0.5), weak adhesion (AC2, 0.5<OD≦1.2), mean adhesion (AC3, 1.2<OD≦2.0) and strong adhesion (AC4, OD>2.0) (Table 3).

To evaluate the effect of enzymatic, physical and chemical treatment on the adhesion ability of the strains, bacterial cultures at the beginning of the stationary phase (6 hrs growth) were submitted to the said treatments at various temperatures and times, thereafter adhesion changes were evaluated. The adhesion properties are reported in the following table. The results show that the adhesion properties of the strains are generally not much altered by physical, chemical and enzymatic treatment.

TABLE 3
Adhesion of L. paracasei IMPC 2.1, compared with L. rhamnosus
GG ATCC53103 and another L. paracasei strain, on an abiotic
surface before and after physical, chemical and enzymatic treatment.

| | Strain | | |
|---|---|---|---|
| | L. | | |
| | rhamnosus | | |
| | GG | L. paracasei | L.paracasei |
| Treatment | ATCC53103 | IMPC 2.1 | IMPC 4.1 |
| Cultures incubated for 24 h | 4 | 4 | 2 |
| in wells | | | |
| Control cells | 4 | 4 | 2 |
| (6 hrs incubation) | | | |
| Physical treatment | | | |
| 30 min/65° C. | 1 | 2 | 1 |
| 15 min/120° C. | 1 | 1 | 1 |
| Enzymatic Treatment | | | |
| Buffer A | 4 | 3 | 2 |
| 5.0 mg/ml trypsin | 1 | 2 | 1 |
| 5.0 mg/ml proteinase | 1 | 2 | 1 |
| 5.0 mg/ml chemotrypsin | 1 | 2 | 1 |
| Buffer B | 4 | 3 | 2 |
| 5.0 mg/ml pepsin | 2 | 3 | 1 |
| Chemical Treatment | | | |
| Buffer C | 4 | 3 | 2 |
| 0.05 M sodium periodate | 4 | 3 | 2 |
| 0.05 M sodium iodate | 4 | 3 | 2 |
| 5M LiCl | 2 | 2 | 1 |

^{a} Adhesion Class (AC):1, OD ≦ 0.5; 2, 0.5 < OD ≦ 1.2; 3, 1.2 < OD ≦ 2.0; 4, OD > 2

Adhesion to Pericarp

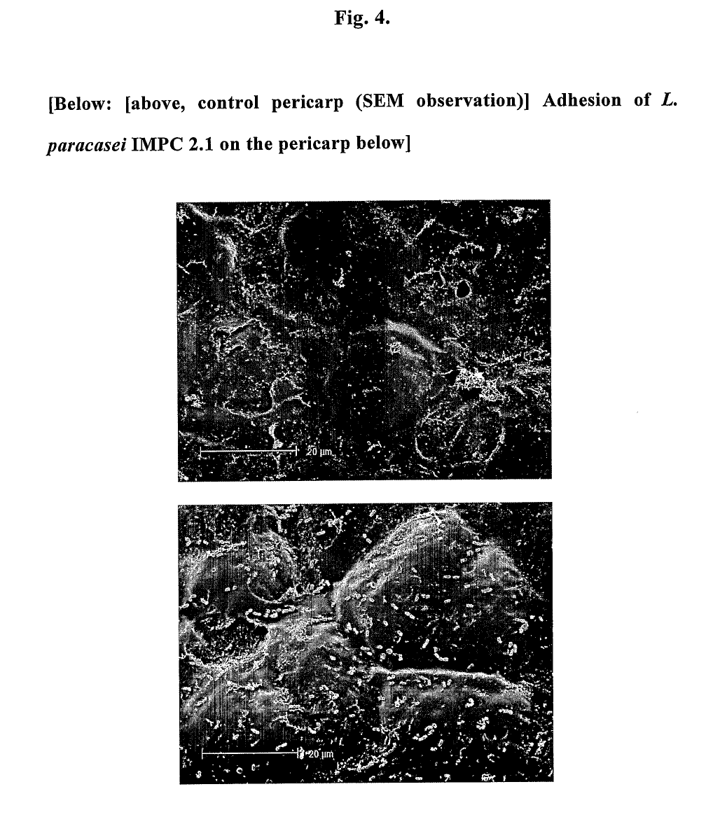

FIG. 4 shows anchoring and distribution of L. paracasei IMPC 2.1 on the pericarp (see also tables 1 and 2).

### Example 3

Persistence of L. paracasei IMPC 2.1 in the Gastro-Intestinal Tract

### Experiment 1

Two healthy adult subjects were fed for 7 days with portions of 5 (subject 1) and 10 (subject 2) olives, thoroughly drained, containing in all 3×10¹⁰ and 6×10¹⁰ UFC of L. paracasei IMPC 2.1 respectively. The composition of the intestinal flora of the subjects was monitored at the beginning (time 0) and after 7 days (t=7) of administration and after 3 days from the end of administration. At each sampling, 1 g of faeces from each subject was added with 9 ml of Amies medium, homogenized and submitted to decimal dilutions, which were plated on a 12 μg/ml Rogosa±vancomycin substrate and cultured under anaerobiosis for 48 hours at 37° C.

TABLE 4
Lactic populations in human subjects before and after
administration of table olives added with L. paracasei IMPC 2.1.

| | Total UFC on Rogosa + vancomycin | | |
|---|---|---|---|
| | | | t = 3 days after |
| | t = 0 | t = days | suspension |
| Subject 1 fed with 3 × 10¹⁰ | 2.7 × 10⁷ | 2 × 10⁹ | 4.5 × 10⁶ |
| UFC/die | | | |
| Subject 2 fed with 6 × 10¹⁰ | 7.0 × 10⁴ | 3.1 × 10⁶ | 5.2 × 10⁵ |
| UFC/die | | | |

An increase of about two logarithmic cycles in the intestinal lactic population of the two subjects was observed; an expected reduction of about 2.5 cycles in subject 1 and of about 1 cycle in subject 2 was observed after suspension of the administration.

Experiment 2

Two healthy adult subjects (A and B) were fed with portions of ten olives containing about 10⁹ CFU of L. paracasei IMPC 2.1. The intestinal microflora of was monitored at the beginning of the experiment (t=0), after 10 days of daily consumption of the product (t=10) and 7 days from the end of administration, according to the procedure described in experiment 1. The results are reported in the following table.

The colonies isolated in both experiments were subjected to molecular identification (see Example 2), whereby it was ascertained that L. paracasei IMPC 2.1 was present in the two subjects and colonized the intestine.

1. Table olives characterized in that they contain lactobacilli and/or bifidobacteria adhering on the pericarp.

2. Olives according to claim 1 characterized in that the lactobacilli are selected from Lactobacillus rhamnosus and L. paracasei and the bifidobacteria are selected from Bifidobacterium bifidum and B. iongum.

3. Table olives according to claim 2 characterized in that the lactobacilli are selected from: Lactobacillus rhamnous GG ATCC53103; L. rhamnosus IMPC 11; L. rhamnosus IMPC 19; Lactobacillus paracasei LMG P-22043; Lactobacillus paracasei IMPC 4.1 and that the bifidobacteria are selected from Bifidobacterium bifidum ATCC15696 and Bifidobacterium longum ATCC15708.

4. Table olives according to claim 3 characterized in that the lactobacilli belong to the strain Lactobacillus paracasei deposited with the Belgian Coordinated Collections of Microorganisms under accession number LMG P-22043.

5. Probiotic food products comprising the table olives of any one of claims 1-4.

6. Use of lactobacilli and bifidobacteria to coat the pericarp of table olives.

7. Lactobacillus paracasei deposited with the Belgian Coordinated Collections of Microorganisms under accession number LMG P-22043.
